(19) United States
(12) Patent Application Publication
(10) Pub. No.: US 2018/0064392 A1
(43) Pub. Date: Mar. 8, 2018

(54) DETECTION OF FOOT EDEMA THROUGH IN-SHOE PRESSURE SENSORS

(71) Applicant: EMPIRE TECHNOLOGY DEVELOPMENT LLC, Wilmington, DE (US)

(72) Inventors: Shmuel Ur, Galil (IL); Noam Hadas, Tel-Aviv (IL)

(73) Assignee: EMPIRE TECHNOLOGY DEVELOPMENT LLC, Wilmington, DE (US)

(21) Appl. No.: 15/258,172

(22) Filed: Sep. 7, 2016

(51) Int. Cl. A61B 5/00; A61B 5/03

(52) U.S. Cl. CPC A61B 5/4878; A61B 5/03; A61B 5/0022; A61B 5/6807; A61B 5/7282

(57) ABSTRACT

Technologies are generally described for detection of foot edema through in-shoe pressure sensors and analysis of detected pressure values. Multiple pressure sensors may be employed in contact with a foot. The pressure sensors may detect pressure generated on a surface of the foot by fluid accumulation within cells of the foot. A change in pressure may be monitored by analyzing pressure values received from the pressure sensors. In some embodiments, a pressure rebound curve may be determined while monitoring the change in the pressure. A comparison between a newly acquired pressure rebound curve and an established-normal pressure rebound curve corresponding to a healthy foot may be made. If the pressure rebound curve is determined to indicate a slower change (rebound) in detected pressure than a change in pressure indicated by the normal pressure rebound curve, the presence of foot edema may be inferred.

### BACKGROUND

Unless otherwise indicated herein, the materials described in this section are not prior art to the claims in this application and are not admitted to be prior art by inclusion in this section.

Foot Edema is a painless swelling which may affect legs, calves or thighs. Because of the eflect of gravity, swelling may particularly be noticeable in the lower part of the body. Foot, leg and ankle swelling may be commonly caused by a blood clot in the leg, a leg infection, veins in the legs that cannot properly pump blood back to the heart, and an injury or surgery involving the leg, ankle, or foot. Swelling may also occur after pelvic surgery, including surgery for pelvic cancer. Long airplane flights or car rides as well as standing for long periods of time may also lead to some swelling in the feet and ankles.

Swelling may also occur in women who take estrogen or during parts of the menstrual cycle. Some women may experience some swelling during pregnancy. More severe swelling during pregnancy may be a sign of preeclampsia (also called toxemia), a serious condition that includes high blood pressure and swelling. Swollen legs may also be a sign of heart failure, kidney failure, or liver failure. In these conditions, there is too much fluid retained in the body. Further, certain medications may also lead to feet edema such as antidepressants, blood pressure medicines, hormones and steroids. Thus, these conditions need early detection, as the risks involved are high.

### SUMMARY

The present disclosure generally describes techniques for detection of foot edema through in-shoe pressure sensors and analysis of detected pressure values.

According to some examples, a system configured to detect foot edema through pressure sensing is described. The system may include a pressure sensor configured to contact a surface of a foot and detect a pressure generated on the surface of the foot by fluid accumulation within cells of the foot; and a processing device communicatively coupled to the pressure sensor. The processing device may include a communication module configured to receive the detected pressure from the pressure sensor; a memory configured to store instructions; and a processor coupled to the communication module and the memory, the processor configured to execute an edema detection application in conjunction with the instructions stored in the memory. The edema detection application may be configured to detect a first transient increase and a subsequent decrease in the detected pressure; monitor a change in the pressure following thee first transient increase and the subsequent decrease in the detected pressure to determine a first pressure rebound curve; compare the first pressure rebound curve to a normal pressure rebound curve, wherein the normal pressure rebound corresponds to a pressure change in a healthy person's foot; and in response to detecting that the first pressure rebound curve indicates a slower increase rate in the detected pressure than a pressure increase rate indicated by the normal pressure rebound curve, infer presence of the foot edema.

According to other examples, a method to detect foot edema through pressure sensing is described. The method may include detecting a first transient increase and a subsequent decrease in a pressure detected through a pressure sensor in contact with a foot, where the pressure is generated on a surface of the foot by fluid accumulation within cells of the foot; monitoring a change in the pressure following the transient increase and the subsequent decrease in the detected pressure to determine a first pressure rebound curve; in response to detecting expiration of a predefined time period following the detection of the first transient increase and the subsequent decrease in the detected pressure, inferring a rest state; in response to inferring the rest state, comparing the first pressure rebound curve to a normal pressure rebound curve, where the normal pressure rebound curve corresponds to a change in detected pressure of a healthy person's foot; and in response to detecting that the first pressure rebound curve indicates a slower increase rate in the detected pressure than a pressure increase rate indicated by the normal pressure rebound curve, infer presence of the foot edema.

According to further examples, an edema detection device configured to detect foot edema through pressure sensing is described. The edema detection device may include a communication module communicatively coupled through a wired or wireless communication medium to one or more pressure sensors each sensor configured to contact a foot and detect a pressure generated on a surface of the foot by fluid accumulation within cells of the foot; a memory configured to store instructions; and a processor coupled to the memory and the communication module. The processor may be configured to detect one or more transient increases and subsequent decreases in the detected pressure through the one or more pressure sensors; determine a pressure rebound curve with each of the one or more pressure sensors based on changes in the pressure following the one or more transient increases and subsequent decreases in the detected pressure; determine a combined pressure rebound curve based on a weighted average of the pressure rebound curves, where the pressure rebound curves are weighted based on locations of corresponding pressure sensors; compare the combined pressure rebound curve to a normal pressure rebound curve, where the normal pressure rebound curve corresponds to a change in pressure of a healthy person's foot; in response to detecting that the combined pressure rebound curve indicates a slower increase rate in the detected pressure than a pressure increase rate indicated by the normal pressure rebound curve, infer presence of the foot edema; and transmit one or more of an audio alert, a visual alert, a tactile alert, and a textual alert to a computing device.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 illustrates an example pressure sensor placed on a foot as part of a system to detect foot edema through in-shoe pressure sensors and analysis of detected pressure values;

FIG. 2 illustrates example pressure sensors placed on different locations of a foot as part of a system to detect foot edema through in-shoe pressure sensors and analysis of detected pressure values;

FIG. 3 illustrates an example system diagram for detection of foot edema through in-shoe wired pressure sensors and analysis of detected pressure values;

FIG. 4 illustrates an example system diagram for detection of foot edema through in-shoe wireless pressure sensors and analysis of detected pressure values;

FIG. 5 illustrates a block diagram of an edema detection device for detection of foot edema through in-shoe wireless pressure sensors and analysis of detected pressure values;

FIG. 6 illustrates an example graphical representation of an example normal pressure rebound curve and an edema-indicating pressure rebound curve following a transient pressure increase and release;

FIG. 7 illustrates a general purpose computing device, which may be associated with detection of foot edema through in-shoe pressure sensors and analysis of detected pressure values;

FIG. 8 is a flow diagram illustrating an example process for detection of foot edema through in-shoe pressure sensors and analysis of detected pressure values, that may be performed by a computing device, such as the computing device in FIG. 7; and

FIG. 9 illustrates a block diagram of an example computer program product, all arranged in accordance with at least some embodiments described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. The aspects of the present disclosure, as generally described herein, and illustrated in the Figures, can be arranged, substituted, combined, separated, and designed in a wide variety of different configurations, all of which are explicitly contemplated herein.

This disclosure is generally drawn, among other things, to methods, apparatus, systems, devices, and/or computer program products related to detection of foot edema through in-shoe pressure sensors and analysis of detected pressure values.

Briefly stated, technologies are generally described for detection of foot edema through in-shoe pressure sensors and analysis of detected pressure values. Multiple pressure sensors may be employed in contact with a foot. The pressure sensors may detect pressure generated on a surface of the foot by fluid accumulation within cells of the foot. A change in pressure may be monitored by analyzing pressure values received from the pressure sensors. In some embodiments, a pressure rebound curve may be determined while monitoring the change in the pressure. A comparison between a newly acquired pressure rebound curve and an established-normal pressure rebound curve corresponding to a healthy foot may be made. If the pressure rebound curve is determined to indicate a slower change (increase rate) in detected pressure than a change in pressure indicated by the normal pressure rebound curve, the presence of foot edema may be inferred.

Because feet edema is related to many health conditions, monitoring of early signs of developing edema may be helpful to take prophylactic or therapeutic actions as early as possible. Such actions may include raising legs above heart level while lying down, exercising, wearing support stockings, or taking appropriate medications.

If edema develops subsequently, it may be useful to a physician to know when the process started, and how quickly it developed. It may also be useful to know if edema affected just one or both feet.

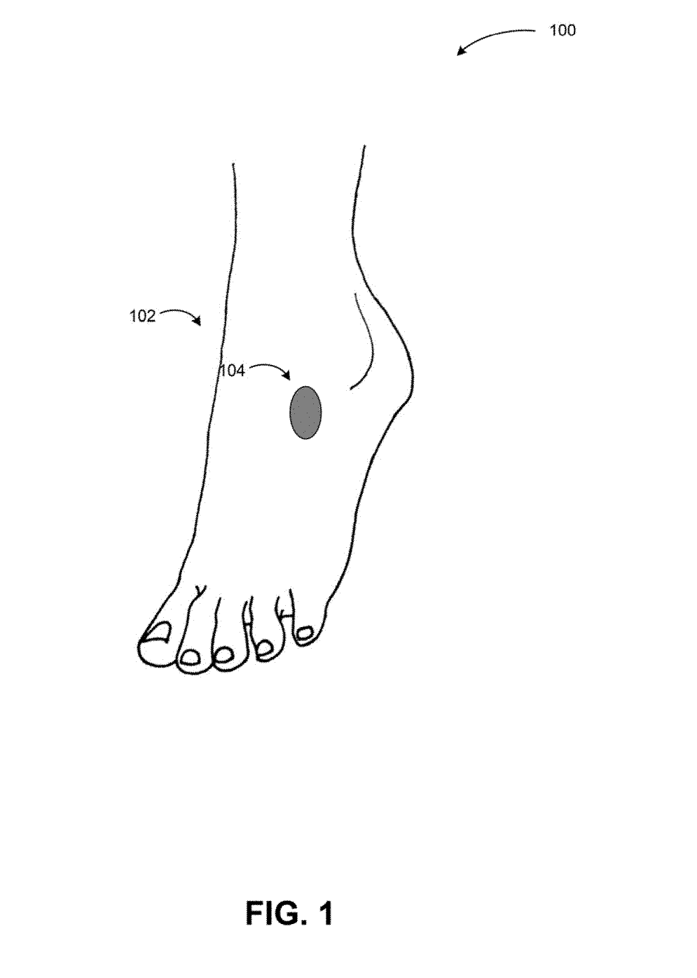

FIG. 1 illustrates an example pressure sensor placed on a foot as part of a system to detect foot edema through in-shoe pressure sensors and analysis of detected pressure values, arranged in accordance with at least some embodiments described herein.

As shown in a diagram 100, a pressure sensor 104 may be placed in contact with a foot 102 to detect foot edema. In some examples, the pressure sensor 104 may be integrated with a shoe. The pressure sensor 104 may be configured to detect a pressure generated on the surface of the foot by fluid accumulation within cells of the foot 102. A baseline pressure level may be assumed, which is due to the sensor pressing against a normal skin tissue.

The skin of the edematous foot contains higher amounts of fluids than normal amounts of fluids, and therefore its volume is larger. Since the fluids may be located between the cells and outside of the blood vessels, these fluids may flow slowly from place to place. When pressure is applied on a specific area of the edematous skin, a dimple forms because these fluids are pushed away from the specific area of the skin. This dimple may fill up again slowly and disappear when pressure is removed, because it takes a relatively longer time for the fluids to flow back to the tissue below the specific area of the skin. This is why this type of edema is also known as “pitting edema”. In contrast, normal skin may dimple less, and the dimple may fill up much more quickly.

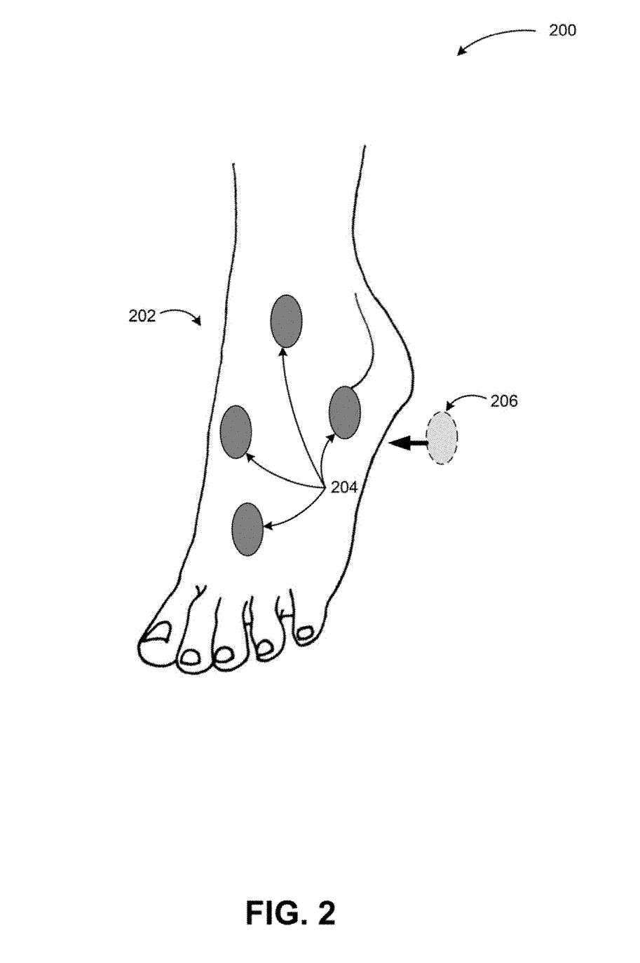

FIG. 2 illustrates example pressure sensors placed on different locations of a foot as part of a system to detect foot edema through in-shoe pressure sensors and analysis of detected pressure values, arranged in accordance with at least some embodiments described herein.

As shown in a diagram 200, multiple pressure sensors 204 may be placed in contact with one or more surfaces of a foot 202 to detect foot edema. The pressure sensors 204 may be placed at selected or random locations on the foot 202. In some examples, the pressure sensors 204 may be integrated with a shoe at different locations. For example, a pressure sensor 206 may be placed under the surface of the foot 202 (e.g., on the sole). The pressure sensors 204 and 206 may sense different pressure values at each location. The sensed pressure values may depend on the mechanical relationship between shoe size and shape to the foot size and shape, but they may also depend on how much tissue is available between the bones and the skin at a particular location (thus, how much fluid can accumulate in the tissue and result in corresponding increase in pressure). The pressure values may be combined, averaged, and/or weighted depending on the locations.

In one example, locations where a larger amount of tissue is available under a corresponding pressure sensor may be assigned a higher pressure variability compared to locations where a smaller amount of tissue is available under a corresponding pressure sensor (e.g., locations over the navicular bone of the foot compared to sole of the foot).

Furthermore, a shape and size of a shoe worn by the person may also impact pressure detection. For example, a tight fitting shoe may result in higher baseline pressure readings compared to a relaxed fitting shoe. Thus, a calibration process may be performed in some embodiments to determine an effect of the shoe on the pressure detection. As described in more detail below, the pressure sensors 204 and 206 may be communicatively coupled to a system to receive and analyze the detected pressure in order to determine a presence of foot edema. For example, the pressure sensors may be wired to a communication or processing module attached to the shoe, wirelessly coupled to the same module or another system not attached to the shoe. A show as used herein refers to any implement wearable on the feet including, but not limited to, boots, sandals, slippers, flip-flops and other foot/leg-protective wear.

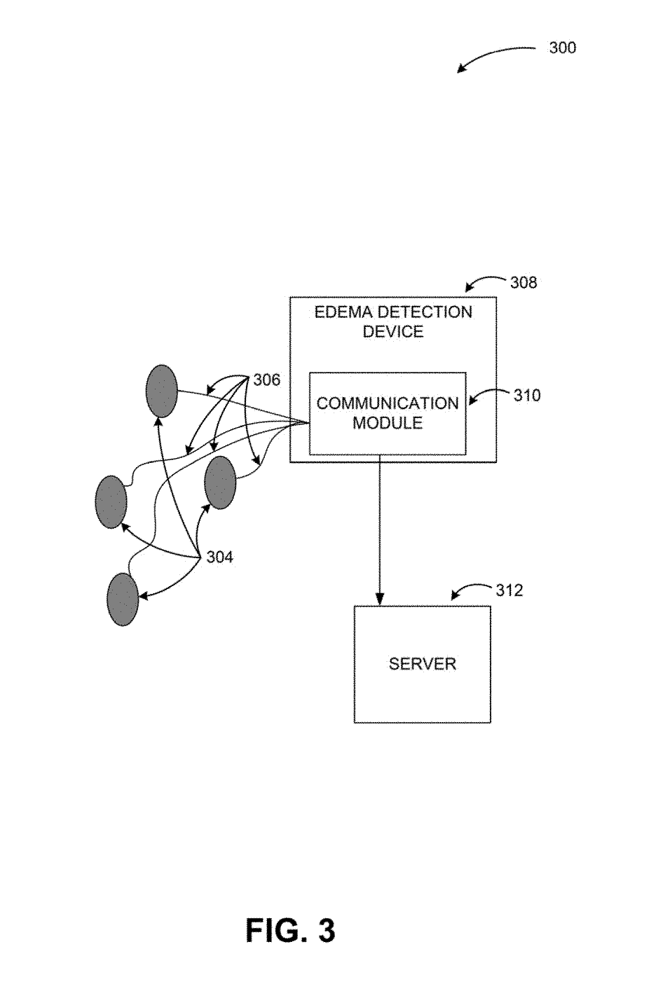

FIG. 3 illustrates an example system diagram for detection of foot edema through in-shoe wired pressure sensors and analysis of detected pressure values, arranged in accordance with at least some embodiments described herein.

As shown in a diagram 300, an edema detection device 308 may be configured to detect foot edema. The edema detection device 308 may include a communication module 310. The communication module 310 may be communicatively connected to multiple pressure sensors 304 through a wired communication medium 306 in some examples. The pressure sensors 304 may be placed at different locations of a foot to detect foot edema. In some examples, the pressure sensors 304 may be integrated with a shoe at different locations. The communication module 310 may receive detected pressure values from the pressure sensors 304 embedded in the shoe. In other examples, at least one of the pressure sensors may include an accelerometer.

During an operation, the pressure sensors 304 may be configured to detect the pressure generated on a surface of the foot due to fluid accumulation within the cells of the foot. The pressure sensors 304 may send the detected pressure values to the communication module 310. The edema detection device 308 may be further configured to analyze the detected pressure values and to monitor multiple transient increases in the detected pressure from the pressure sensors. The edema detection device 308 may be further configured to determine multiple pressure rebound curves based on changes in the pressure from the different pressure sensors. A combined pressure rebound curve may be determined based on a combination (e.g., an average) of the multiple pressure rebound curves. A comparison between a determined pressure rebound curve and a normal pressure rebound curve may be performed. If the determined pressure rebound curve indicates a slower change (increase rate) in the detected pressure than a rebound indicated by the normal pressure rebound curve, the presence of foot edema may be inferred. If the determined pressure rebound curve indicates a faster change (increase rate) in the detected pressure than the rebound indicated by the nonnal pressure rebound curve, a (re-)calibration of the normal pressure rebound curve may be performed based on the faster pressure rebound curve.

In other examples, the edema detection device 308 may be further configured to adjust a pressure rebound curve based on one or more parameters associated with a person on whose foot is being monitored. The parameters may include, for example, a weight of the person, a size of the foot of the person, an average blood pressure level of the person, and a health indicator of the person, among others.

The communication module 310 may be further configured to transmit data associated with the detected pressure or determined pressure rebound curve to a server 312. The transmitted data may include raw detected pressure data, determined pressure rebound curve, comparison results, and/or other data such as analysis results of the pressure rebound curve. The edema detection device 308 may not necessarily be in contact with the foot. For example, the edema detection device 308 may be a portable device attached to the shoe. The portable device could be on straps of the shoe, or in walls of the shoe. Furthermore, the pressure sensors 304 may also be in direct or indirect (e.g., through a pressure sensitive material) contact with the surface of the foot.

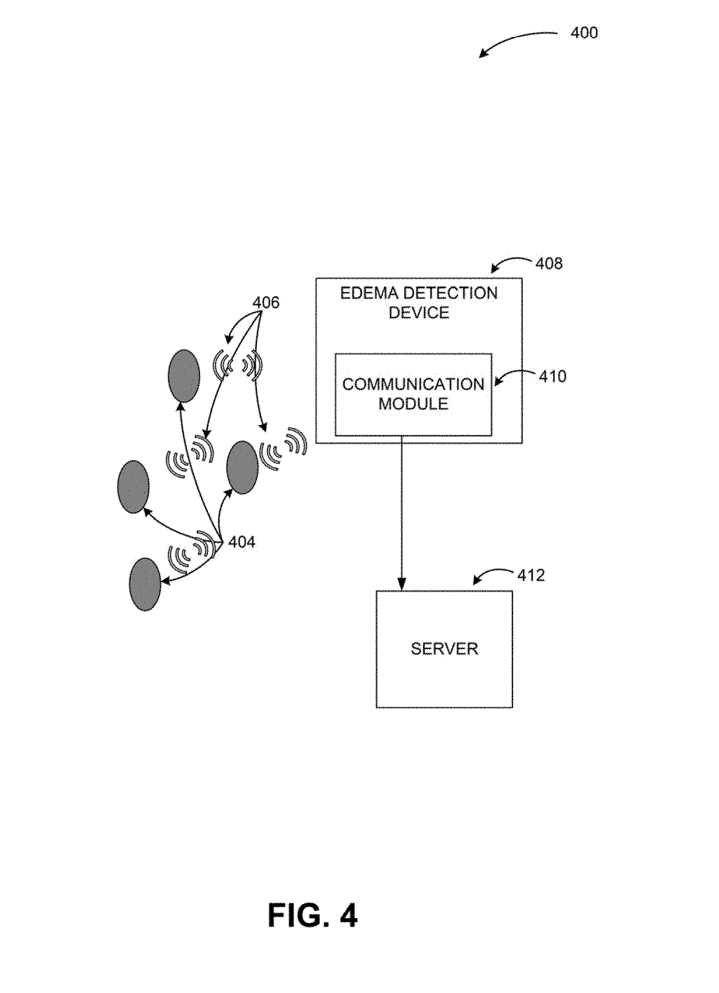

FIG. 4 illustrates an example system diagram for detection of foot edema through in-shoe wireless pressure sensors and analysis of detected pressure values, arranged in accordance with at least some embodiments described herein.

As shown in a diagram 400, an edema detection device 408 may be configured to detect foot edema. The edema detection device 408 may include a communication module 410. The communication module 410 may be communicatively connected to multiple pressure sensors 404 through a wireless communication medium 406. The communication module 410 receive the detected pressure values from the pressure sensors 404 embedded in the shoe at different locations. The wireless communication medium may be a radio frequency (RF) communication such as Bluetooth or a Wi-Fi technology, an infrared communication medium, a visible light communication medium, or one based on magnetic or electric fields, as well as acoustic communication. Furthermore, the wireless communication may be direct, that is, directly between the pressure sensors and the edema detection device 408 or indirect, that is via intermediary communication devices or media (e.g., Internet communication or via a local router).

In some embodiments, the communication module 410 may be further configured to transmit an alert to a server 412 or a communication device associated with the person (e.g, a smart phone) in response to inferring the presence of the foot edema. The alert may be an audio alert, a visual alert, a tactile alert, or a textual alert. In ether embodiments, the edema detection device 408 may be a portable or wearable device worn or carried by the person, a special purpose computer or a general purpose computer within the person's home or office, or a remotely placed device, for example, at a health care provider's, facility.

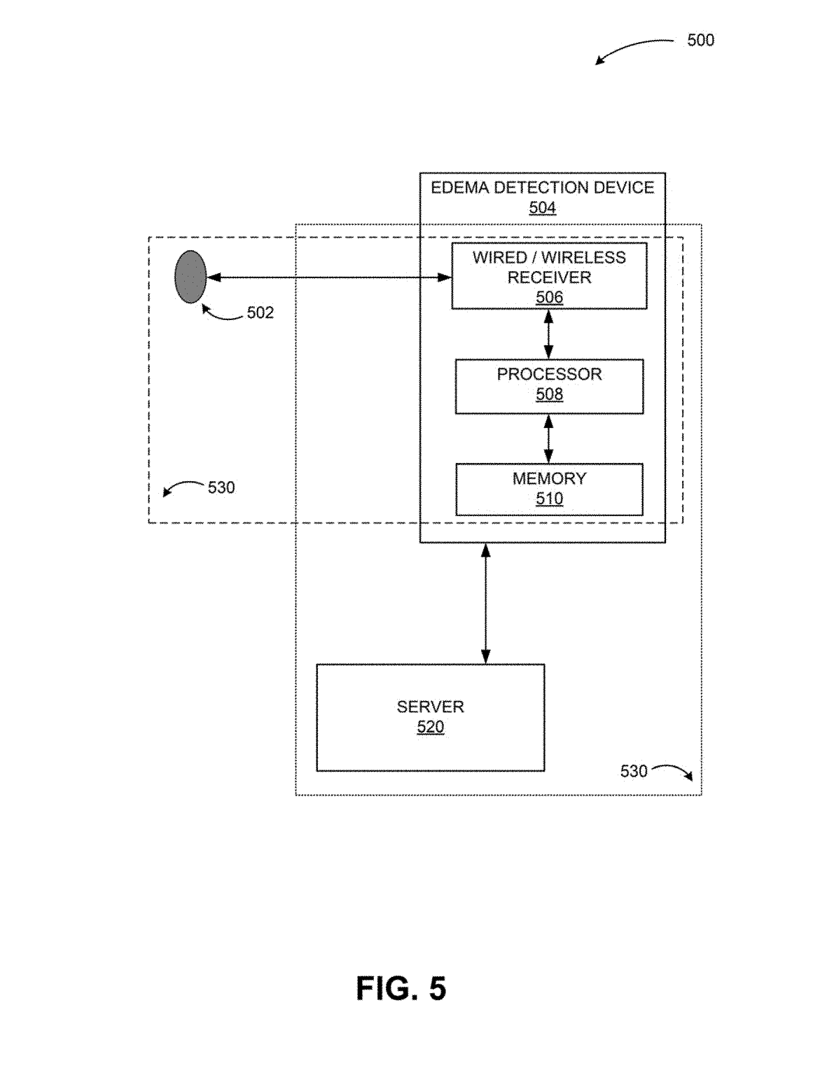

FIG. 5 illustrates a block diagram of an edema detection device for detection of foot edema through in-shoe wireless pressure sensors and analysis of detected pressure values, arranged in accordance with at least some embodiments described herein.

As shown in a diagram 500, an edema detection device 504 may be configured to detect foot edema as described above. The edema detection device 504 may include a wired/wireless receiver 506, a memory 510 configured to store instructions, and a processor 508 coupled to the memory and the wired/wireless receiver 506. The wired/wireless receiver 506 may be communicatively coupled to a pressure sensor 502. The pressure sensor 502 may be configured to contact a foot and detect a pressure generated on a surface of the foot by fluid accumulation within cells of the foot.

During an operation, the pressure sensor 502 may send detected pressure values to the wired/wireless receiver 506. The processor 508 may determine a state of activity by monitoring a transient increase in the detected pressure. It may infer a rest state if a predefined time period expires following the detection of a first transient increase in the detected pressure. Otherwise, the processor 508 may infer an activity state and disregard detected pressure values. In some embodiments, a pressure rebound curve may be determined while monitoring the change in the pressure. A comparison between the determined pressure rebound curve and a normal pressure rebound curve corresponding to a healthy foot in rest state may be made. If the determined pressure rebound curve in the rest state indicates a slower increase rate or rebound in the detected pressure than an increase rate or rebound indicated by the normal pressure rebound curve, the presence of foot edema may be inferred.

The edema detection device 504 may transmit the inference of presence of foot edema to a server 520. The server 520 may further transmit one or more of an audio alert, a visual alert, a tactile alert, and a textual alert to a computing device.

In some embodiments, the processor 508, the memory 510, the wired/wireless receiver 506, and/or the pressure sensor 502 may be a separate portable device (as indicated by the dashed lines 530) attached to the shoe or carried on the body of the user. The portable device may be attached to straps of the shoe, or to a wall of the shoe, for example.

In other embodiments, the pressure sensor 502 may be physically separate from the edema detection device 504. In further embodiments, the processor 508, the memory 510, and/or the wired/wireless receiver 506 may be integrated components of a server 520 (as indicated by the dotted lines 540) and receive data associated with detected pressure directly from the pressure sensor 502 or via the edema detection device 504. Thus, components and functionality of a system according to embodiments may be distributed in various ways among portable/wearable devices and remote computing devices.

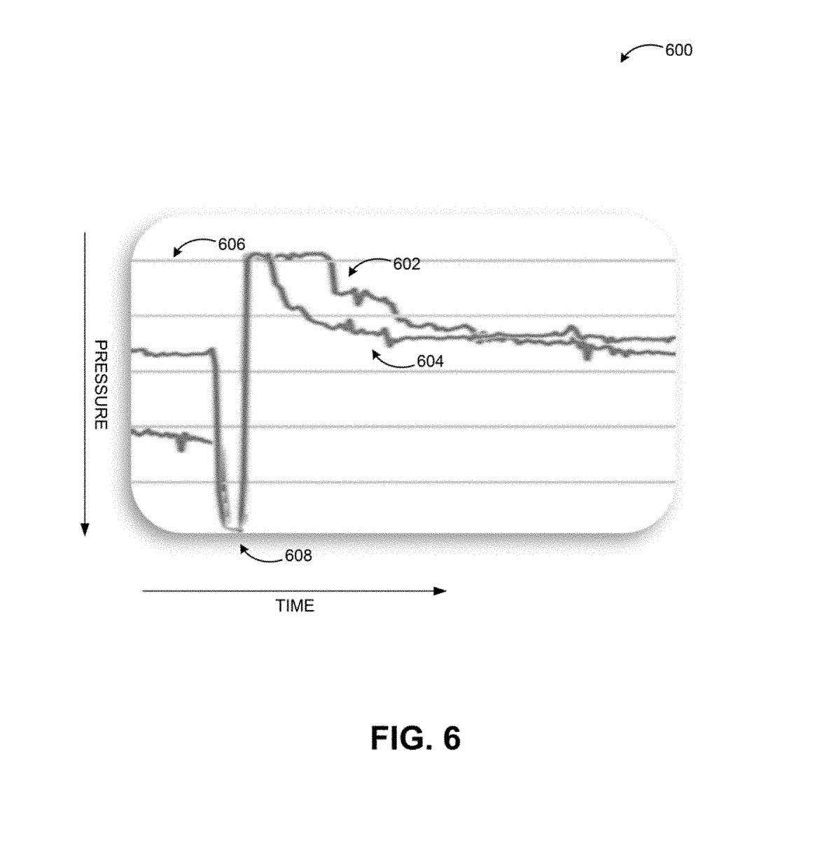

FIG. 6 illustrates an example graphical representation of an example normal pressure rebound curve and an edema indicating pressure rebound curve following a transient pressure increase and decrease, arranged in accordance with at least some embodiments described herein.

A graph 600 depicts pressure characteristics along pressure and time axes. In one scenario, following a transient increase 608, a relatively short relay and a return to normal, a pressure rebound curve 602 may indicate a slower increase rate of detected pressure values from a minimum value 606 compared to a pressure rebound curve 604, where the detected pressure values rebound relatively faster. Thus, if the pressure rebound curve 604 is the normal pressure rebound curve (corresponding to healthy person in rest state), the pressure rebound curve 602 may indicate presence of foot edema.

Because detected pressure on the foot surface(s) may change frequently and randomly in an activity state (e.g., when the person is walking, running, dancing, or performing other physical activities), detected pressure values may not be useful in such a state. However, the activity state may be inferred from the changes, that is, the transient increase being repeated within a predefined time period. The time period (threshold for rest state) may be determined from calibration measurements in some examples and may range from 30 seconds to several minutes, for example.

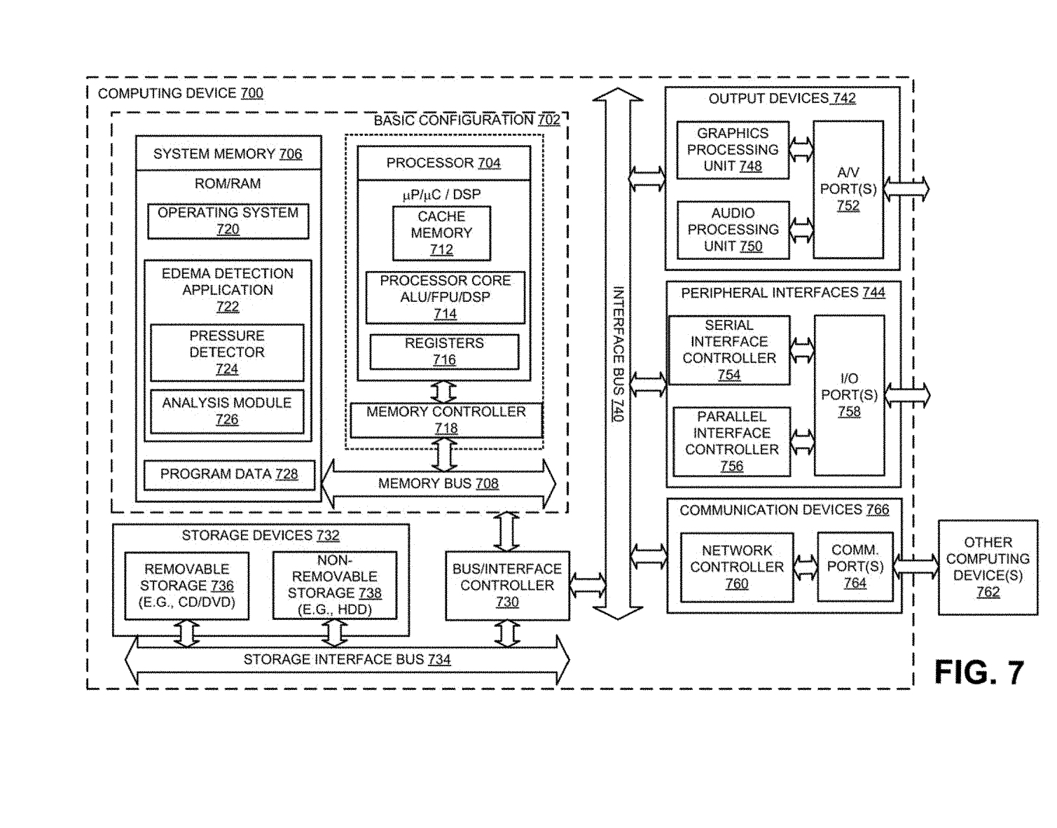

FIG. 7 illustrates a general purpose computing device, which may be associated with detection of foot edema through in-shoe pressure sensors and analysis of detected pressure values, arranged in accordance with at least some embodiments described herein.

For example, the computing device 700 may be a management or security server at a datacenter associated with detection of foot edema through in-shoe pressure sensors and analysis of detected pressure values as described herein. In an example basic configuration 702, the computing device 700 may include one or more processors 704 and a system memory 706. A memory bus 708 may be used to communicate between the processor 704 and the system memory 706. The basic configuration 702 is illustrated in FIG. 7 by those components within the inner dashed line.

Depending on the desired configuration, the processor 704 may be of any type, including but not limited to a microprocessor (μP), a microcontroller (μC), a digital signal processor (DSP), or any combination thereof. The processor 704 may include one more levels of caching, such as a cache memory 712, a processor core 714, and registers 716. The example processor core 714 may include an arithmetic logic unit (ALU), a floating point unit (FPU), a digital signal processing core (DSP Core), or any combination thereof. An example memory controller 718 may also be used with the processor 704, or in some implementations, the memory controller 718 may be an internal part of the processor 704.

Depending on the desired configuration, the system memory 706 may be of any type including but not limited to volatile memory (such as RAM), non-volatile memory (such as ROM, flash memory, etc.) or any combination thereof. The system memory 706 may include an operating system 720, edema detection application 722, and program data 728. The edema detection application 722 may include a pressure detector 724 and an analysis module 726 for detection of foot edema through in-shoe pressure sensors and analysis of detected pressure values as described herein.

The computing device 700 may have additional features or functionality, and additional interfaces to facilitate communications between the basic configuration 702 and any desired devices and interfaces. For example, a bus/interface controller 730 may be used to facilitate communications between the basic configuration 702 and one or more data storage devices 732 via a storage interface bus 734. The data storage devices 732 may be one or more removable storage devices 736, one or more non-removable storage devices 738, or a combination thereof. Examples of the removable storage and the non-removable storage devices include magnetic disk devices such as flexible disk drives and hard-disk drives (HDDs), optical disk drives such as compact disc (CD) drives or digital versatile disk (DVD) drives, solid state drives (SSDs), and tape drives to name a few. Example computer storage media may include volatile and nonvolatile, removable and non-removable media implemented in any method or technology for storage of information, such as computer readable instructions, data structures, program modules, or other data.

The system memory 706, the removable storage devices 736 and the non-removable storage devices 738 are examples of computer storage media. Computer storage media includes, but is not limited to, RAM, ROM, EEPROM, flash memory or other memory technology, CD-ROM, digital versatile disks (DVDs), solid state drives, or other optical storage, magnetic cassettes, magnetic tape, magnetic disk storage or other magnetic storage devices, or any other medium which may be used to store the desired information and which may be accessed by the computing device 700. Any such computer storage media may be part of the computing device 700.

The computing device 700 may also include an interface bus 740 for facilitating communication from various interface devices (for example, one or more output devices 742, one or more peripheral interfaces 744, and one or more communication devices 766) to the basic configuration 702 via the bus/interface controller 730. Some of the example output devices 742 include a graphics processing unit 748 and an audio processing unit 750, which may be configured to communicate to various external devices such as a display or speakers via one or more A/V ports 752. One or more example peripheral interfaces 744 may include a serial interface controller 754 or a parallel interface controller 756, which may be configured to communicate with external devices such as input devices (for example, keyboard, mouse, pen, voice input device, touch input device, etc.) or other peripheral devices (for example, printer, scanner, etc.) via one or more I/O ports 758. An example communication device 766 includes a network controller 760, which may be arranged to facilitate communications with one or more other computing devices 762 over a network communication link via one or more communication ports 764.

The network communication link may be one example of a communication media. Communication media may be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR) and other wireless media. The term computer readable media as used herein may include both storage media and communication media

The computing device 700 may be implemented as a part of a general purpose or specialized server, mainframe, or similar computer that includes any of the above functions. The computing device 700 may also be implemented as a personal computer including both laptop computer and non-laptop computer configurations

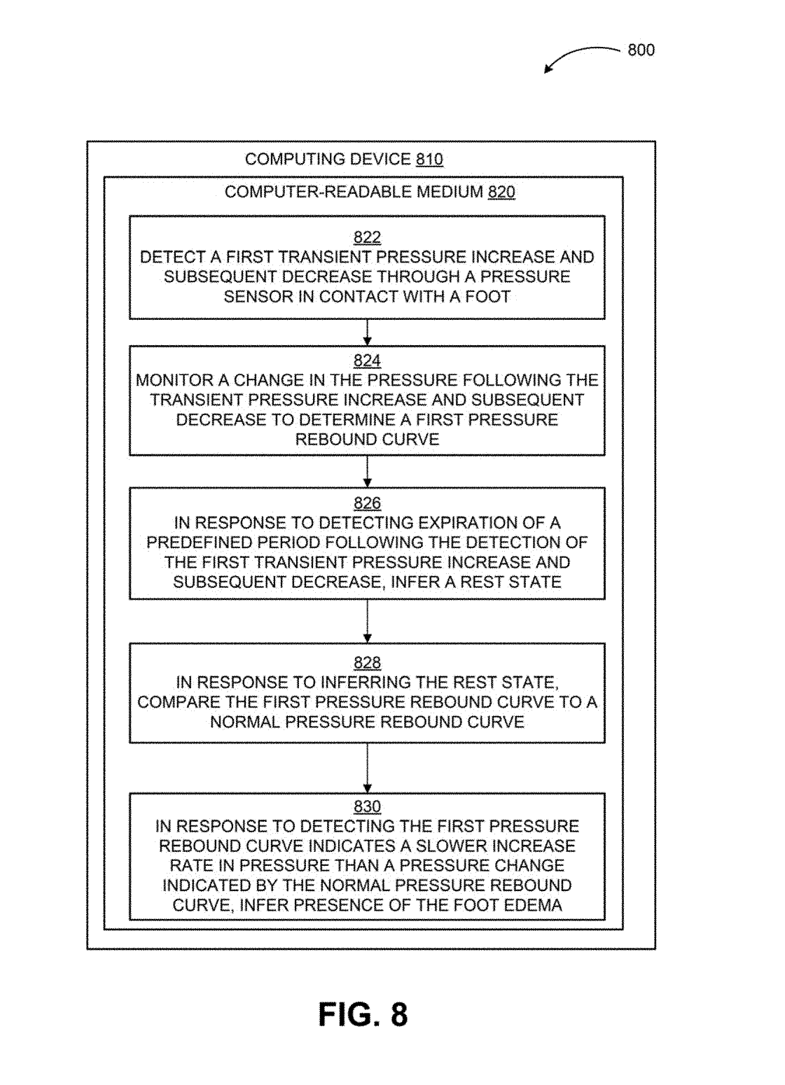

FIG. 8 is a flow diagram illustrating an example process for detection of foot edema through in-shoe pressure sensors and analysis of detected pressure values, that may be performed by a computing device, such as the computing device, in FIG. 7, arranged in accordance with at least some embodiments described herein.

Example methods may include one or more operations, functions or actions as illustrated by one or more of blocks 822, 824, 826, 828 and/or 830, and may in some embodiments be performed by a computing device such as the computing device 700 in FIG. 7. The operations described in the blocks 822-830 may also be stored as computer-executable instructions in a computer-readable medium such as a computer-readable medium 820 of a computing device 810.

An example process to detect foot edema through pressure sensing may begin with block 822, “DETECT A FIRST TRANSIENT INCREASE AND SUBSEQUENT DECREASE IN A PRESSURE DETECTED THROUGH A PRESSURE SENSOR IN CONTACT WITH A FOOT”, where an edema detection application implemented on a server may detect a first transient increase and subsequent decrease in a pressure through a pressure sensor in contact with a foot. In some embodiments, the pressure may be generated on a surface of the foot by fluid accumulation within cells of the foot.

Block 822 may be followed by block 824, “MONITOR A CHANGE IN THE PRESSURE FOLLOWING THE TRANSIENT INCREASE AND SUBSEQUENT DECREASE IN THE DETECTED PRESSURE TO DETERMINE A FIRST PRESSURE REBOUND CURVE”, where edema detection application implemented on a server may monitor a change in the pressure because of transient increase and subsequent decrease in the detected pressure. In some embodiments, a pressure rebound curve may be determined while monitoring the change in the pressure. The pressure rebound curve may correspond to a change in pressure indicating a return of the skin to its normal shape over time after the transient pressure event.

Block 824 may be followed by block 826, “IN RESPONSE TO DETECTING EXPIRATION OF A PREDEFINED TIME PERIOD FOLLOWING THE DETECTION OF THE FIRST TRANSIENT INCREASE AND SUBSEQUENT DECREASE IN THE DETECTED PRESSURE, INFER A REST STATE”, where edema detection application may detect expiration of a predefined period after detecting an increase in transient pressure and subsequent decrease. In some embodiments, the edema detection application may infer a rest state after detecting the expiration of the predefined period.

Block 826 may be followed by block 828, “IN RESPONSE TO INFERRING THE REST STATE, COMPARE THE FIRST PRESSURE REBOUND CURVE TO A NORMAL PRESSURE REBOUND CURVE”, where edema detection application may compare the first pressure rebound curve to a normal pressure rebound curve after getting inference of the rest state.

Block 828 may be followed by block 830, “IN RESPONSE TO DETECTING THE FIRST PRESSURE REBOUND CURVE INDICATES A SLOWER INCREASE RATE IN PRESSURE THAN A PRESSURE CHANGE INDICATED BY THE NORMAL PRESSURE REBOUND CURVE, INFER PRESENCE OF THE FOOT EDEMA”, where edema detection application may infer presence of the foot edema in response to detecting that first pressure rebound curve indicates a slower increase rate in the detected pressure than an increase rate indicated by the normal pressure rebound curve. The inference of foot edema may be followed by transmission of an audio alert, a visual alert, a tactile alert, and/or a textual alert to a computing device.

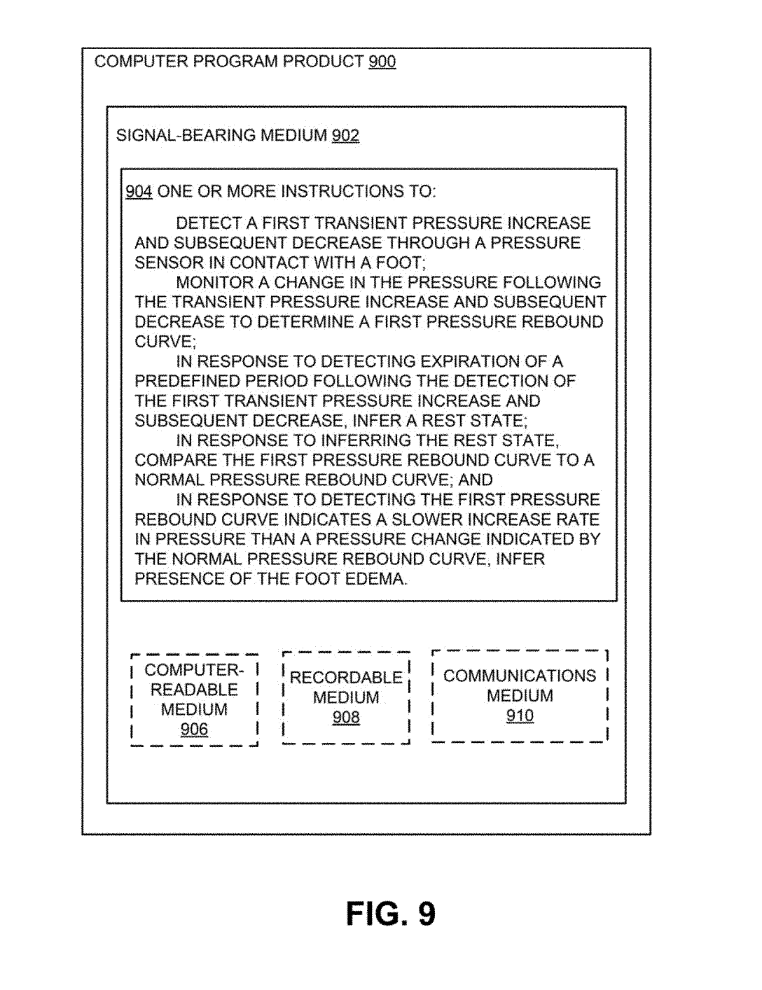

FIG. 9 illustrates a block diagram of an example computer program product, arranged in accordance with at least some embodiments described herein.

In some examples, as shown in FIG. 9, a computer program product 900 may include a signal-bearing medium 902 that may also include one or more machine readable instructions 904 that, when executed by, for example, a processor may provide the functionality described herein. Thus, for example, referring to the processor 704 in FIG. 7, the edema detection application 722 may undertake one or more of the tasks shown in FIG. 9 in response to the instructions 904 conveyed to the processor 704 by the signal-bearing medium 902 to perform actions associated with detecting of foot edema through in-shoe pressure sensors as described herein. Some of those instructions may include, for example, instructions to detect a first transient increase and subsequent decrease in a pressure detected through a pressure sensor in contact with a foot, where the pressure is generated on a surface of the foot by fluid accumulation within cells of the foot, monitor a change in the pressure following the transient increase and subsequent decrease in the detected pressure to determine a first pressure rebound curve, infer a rest state in response to detecting expiration of a predefined time period following the detection of the first transient and subsequent decrease increase in the detected pressure, compare the first pressure rebound curve to a normal pressure rebound curve in response to inferring the rest state, and/or infer presence of the foot edema in response to detecting that the first pressure rebound curve indicates a slower increase rate in the detected pressure than a change indicated by the normal pressure rebound curve, according to some embodiments described herein.

In some implementations, the signal-bearing medium 902 depicted in FIG. 9 may encompass computer-readable medium 906, such as, but not limited to, a hard disk drive, a solid state drive, a Compact Disc (CD), a Digital Versatile Disk (DVD), a digital tape memory, etc. In some implementations, the signal-bearing medium 902 may encompass recordable medium 908, such as, but not limited to, memory, read/write (R/W) CDs, R/W DVDs, etc. In some implementations, the signal-bearing medium 902 may encompass communications medium 910, such as, but not limited to, a digital and/or an analog communication medium (for example, a fiber optic cable, a waveguide, a wired communications link, a wireless communication link, etc.). Thus, for example, the computer program product 900 may be conveyed to one or more modules of the processor 704 by an RF signal bearing medium, where the signal-bearing medium 902 is conveyed by a communications medium 910 (for example, a wireless communications medium conforming with the IEEE 802.11 standard).

According to some examples, a system configured to detect foot edema through pressure sensing is described. The system may include a pressure sensor configured to contact a surface of a foot and detect a pressure generated on the surface of the foot by fluid accumulation within cells of the foot; and a processing device communicatively coupled to the pressure sensor. The processing device may include a communication module configured to receive the detected pressure from the pressure sensor; a memory configured to store instructions; and a processor coupled to the communication module and the memory, the processor configured to execute an edema detection application in conjunction with the instructions stored in the memory. The edema detection application may be configured to detect a first transient increase and subsequent decrease in the detected pressure; monitor a change in the pressure following the first transient increase and the subsequent decrease in the detected pressure to determine a first pressure rebound curve; compare the first pressure rebound curve to a normal pressure rebound curve, wherein the normal pressure rebound curve corresponds to a pressure change in a healthy person's foot; and in response to detecting that the first pressure rebound curve indicates a slower increase rate in the detected pressure than a pressure increase rate indicated by the normal pressure rebound curve, infer presence of the foot edema.

According to other examples, the edema detection application may be further configured to detect a second transient increase and subsequent decrease in the detected pressure within a predefined time period following the first transient increase and the subsequent decrease in the detected pressure; in response to detecting the second transient increase and the subsequent decrease in the detected pressure, infer an activity state; ignore the first pressure rebound curve; and compare the second pressure rebound curve to the normal pressure rebound curve. The edema detection application may also be configured to infer the activity state based on input from at least one other sensor. The at least one other sensor may include an accelerometer. The edema detection application may be further configured to in response to detecting expiration of a predefined time period following the detection of the first transient increase and the subsequent decrease in the detected pressure, infer a rest state; and compare the first pressure rebound curve to the normal pressure rebound currve in response to inferring the rest state.

According to further examples, the edema detection application may be further configured to in response to inferring the presence of the foot edema, transmit an alert to a computing device. The alert may be one or more of an audio alert, a visual alert, a tactile alert, and a textual alert. The system may further include a plurality of pressure sensors each sensor configured to contact one or more surfaces of the foot, and the edema detection application may be further configured to detect a plurality of transient increases and subsequent decreases in the detected pressure from the plurality of pressure sensors; determine a plurality of pressure rebound curves based on changes in the pressure following the plurality of transient increases and the subsequent decreases in the detected pressure; determine a combined pressure rebound curve based on an average of the plurality of pressure rebound curves; compare the combined pressure rebound curve to the normal pressure rebound curve; and in response to detecting that the combined pressure rebound curve indicates a slower increase rate in the detected pressure than a pressure increase rate indicated by the normal pressure rebound curve, infer presence of the foot edema.

According to yet other examples, the edema detection application may be further configured to weight one or more of the plurality of pressure rebound curves to determine the combined pressure rebound curve and weight the one or more of the plurality of pressure rebound curves based on locations of one or more corresponding pressure sensors. The system may also include a plurality of pressure sensors each sensor configured to contact one or more surfaces of the foot, and the edema detection application may be further configured to detect a plurality of transient increases and subsequent decreases in the detected pressure from the plurality of pressure sensors; determine a plurality of pressure rebound curves based on changes in the detected pressure following the plurality of transient pressure increases and the subsequent decreases; and select a pressure rebound curve of the plurality of pre sure rebound curves that indicates a fastest rebound in the detected pressure as the first pressure rebound curve.

According to other examples, a method to detect foot edema through pressure sensing is described. The method may include detecting a first transient increase and a subsequent decrease in a pressure detected through a pressure sensor in contact with a foot, where the pressure is generated on a surface of the foot by fluid accumulation within cells of the foot; monitoring a change in the pressure following the transient increase and the subsequent decrease in the detected pressure to determine a first pressure rebound curve; in response to detecting expiration of a predefined time period following the detection of the first transient increase and the subsequent decrease in the detected pressure, inferring a rest state; in response to inferring the rest state, comparing the first pressure rebound curve to a normal pressure rebound curve, where the normal pressure rebound curve corresponds to a change in detected pressure of a healthy person's foot; and in response to detecting that the first pressure rebound curve indicates a slower increase rate in the detected pressure than a pressure increase rate indicated by the normal pressure rebound curve, infer presence of the foot edema.

According to further examples, the method may also include in response to inferring the presence of the foot edema, transmitting one or more of an audio alert, a visual alert, a tactile alert, and a textual alert to a computing device. The method may further include determining that one or more pressure rebound curves indicate a faster increase rate in the detected pressure than the pressure increase rate indicated by the normal pressure rebound curve in the rest state; and calibrating the normal pressure rebound curve based on the one or more pressure rebound curves. The method may also include adjusting the first pressure rebound curve based on one or more parameters associated with a person on whose foot the first transient increase and the subsequent decrease is detected.

According to some examples, the one or more parameters may include one or more of: a weight of the person, a size of the foot of the person, an average blood pressure level of the person, and a health indicator of the person. The method may also include detecting a plurality of transient increases and subsequent decreases in the detected pressure through a plurality of pressure sensors in contact with the foot; determining a plurality of pressure rebound curves based on changes in the pressure following the plurality of transient increases and the subsequent decreases in the detected pressure; determining a combined pressure rebound curve based on a weighted average of the plurality of pressure rebound curves, where the one or more of the plurality of pressure rebound curves are weighted based on locations of corresponding pressure sensors; comparing the combined pressure rebound curve to the normal pressure rebound curve; and in response to detecting that the combined pressure rebound curve indicates a slower increase rate in the detected pressure than the pressure increase rate indicated by the normal pressure rebound curve, inferring presence of the foot edema.

According to further examples, an edema detection device configured to detect foot edema through pressure sensing is described. The edema detection device may include a communication module communicatively coupled through a wired or wireless communication medium to one or more pressure sensors each sensor configured to contact a foot and detect a pressure generated on a surface of the foot by fluid accumulation within cells of the foot; a memory configured to store instructions; and a processor coupled to the memory and the communication module. The processor may be configured to detect one or more transient increases and subsequent decreases in the detected pressure through the one or more pressure sensors; determine a pressure rebound curve with each of the one or more pressure sensors based on changes in the pressure following the one or more transient increases and subsequent decreases in the detected pressure; determine a combined pressure rebound curve based on a weighted average of the pressure rebound curves, where the pressure rebound curves are weighted based on locations of corresponding pressure sensors; compare the combined pressure rebound curve to a normal pressure rebound curve, where the normal pressure rebound curve corresponds to a change in pressure of a healthy person's foot; in response to detecting that the combined pressure rebound curve indicates a slower increase rate in the detected pressure than a pressure increase rate indicated by the normal pressure rebound curve, infer presence of the foot edema; and transmit one or more of an audio alert, a visual alert, a tactile alert, and a textual alert to a computing device.

According to yet other examples, the edema detection device may be portable device attachable to a shoe. The edema detection device may also be integrated with a shoe.

Various embodiments may be implemented in hardware, software, or combination of both hardware and software (or other computer-readable instructions stored on a non-transitory computer-readable storage medium and executable by one or more processors); the use of hardware or software is generally (but not always, in that in certain contexts the choice between hardware and software may become significant) a design choice representing cost vs. efficiency tradeoffs. There are various vehicles by which processes and/or systems and/or other technologies described herein may be effected (for example, hardware, software, and/or firmware), and the preferred vehicle will vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, each function and/or operation within such block diagrams, flowcharts, or, examples may be implemented, individually and/or collectively, by a wide range of hardware, software, firmware, or virtually any combnation thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, some aspects of the embodiments disclosed herein, in whole or in part, may be equivalently implemented in integrated circuits, as one or more computer programs executing on one or more computers (for example, as one or more programs executing on one or more computer systems), as one or more programs executing on one or more processors for example, as one or more programs executing on one or more microprocessors), as firmware, or as virtually any combination thereof, and designing the circuitry and/or writing the code for the software and/or firmware are possible in light of this disclosure.

The present disclosure is not to be limited in terms of the particular embodiments described in this application, which are intended as illustrations of various aspects. Many modifications and variations can be made without departing from its spirit and scope. Functionally equivalent methods and apparatuses within the scope of the disclosure, in addition to those enumerated herein, are possible from the foregoing descriptions. Such modifications and variations are intended to fall within the scope of the appended claims. The present disclosure is to be limited only by the terms of the appended claims, along with the full scope of equivalents to which such claims are entitled. Also, the terminology used herein is for the purpose of describing particular embodiments only, and is not intended to be limiting.

In addition, the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Versatile Disk (DVD), a digital tape, a computer memory, a solid state drive, etc.; and a transmission type medium such as a digital and/or an analog communication medium (for example, a fiber optic cable, a waveguide, a wired communications link, a wireless communication link, etc.),

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein may be integrated into a data processing system via a reasonable amount of experimentation. A data processing system may include one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (for example, feedback for sensing position and/or velocity of gantry systems; control motors to move and/or adjust components and/or quantities).

A data processing system may be implemented utilizing any suitable commercially available components, such as those found in data computing/communication and/or network computing/communication systems. The herein described subject matter sometimes illustrates different components contained within, or connected with, difIerent other components. Such depicted architectures are merely exemplary, and in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermediate components. Likewise, any two components so associated may also be viewed as being “operably connected”, or “operably coupled”, to each other to achieve the desired functionality, and any two components capable of being so associated may also be viewed as being “operably couplable”, to each other to achieve the desired functionality. Specific examples of operably couplable include but are not limited to physically connectable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations).

Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or ,5 cells, and, so forth.

While various aspects and embodiments have been disclosed herein, other aspects and embodiments are possible. The various aspects and embodiments disclosed herein are for purposes of illustration and are not intended to be limiting, with the true scope and spirit being indicated by the following claims.

What is claimed is:

1. A system configured to detect foot edema through pressure sensing, the system comprising:
a pressure sensor configured to contact a surface of a foot, the pressure sensor further configured to detect a pressure generated on the surface of the foot by fluid accumulation within cells of the foot; and
a processing device communicatively coupled to the pressure sensor, the processing device comprising:
a communication module configured to receive the detected pressure from the pressure sensor;
a memory configured to store instructions; and
a processor coupled to the communication module and the memory, the processor configured to execute an edema detection application in conjunction with the instructions stored in the memory, wherein the edema detection application is configured to:
detect a first transient increase and a subsequent decrease in the detected pressure;
monitor a change in the pressure following the first transient increase and the subsequent decrease in the detected pressure to determine a first pressure rebound curve;
compare the first pressure rebound curve to a normal pressure rebound curve, wherein the normal pressure rebound curve corresponds to a pressure change in a healthy person's foot; and
in response to detecting that the first pressure rebound curve indicates a slower increase rate in the detected pressure than a pressure increase rate indicated by the normal pressure rebound curve, infer presence of the foot edema.

2. The system of claim 1, wherein the edema detection application is further configured to:
detect a second transient increase and subsequent decrease in the detected pressure within a predefined time period following the first transient increase and the subsequent decrease in the detected pressure;
in response to detecting the second transient increase and the subsequent decrease in the detected pressure, infer an activity state;
ignore the first pressure rebound curve; and
compare the second pressure rebound curve to the normal pressure rebound curve.

3. The system of claim 2, wherein the edema detection application is further configured to:
infer the activity state based on input from at least one other sensor.

4. The system of claim 3, wherein the at least one other sensor includes an accelerometer.

5. The system of claim 1, wherein the edema detection application is further configured to:
in response to detecting expiration of a predefined time period following the detection of the first transient increase and the subsequent decrease in the detected pressure, infer a rest state; and
compare the first pressure rebound curve to the normal pressure rebound curve in response to inferring the rest state.

6. The system of claim 1, wherein the edema detection application is further configured to:
in response to inferring the presence of the foot edema, transmit an alert to a computing device.

7. The system of claim 6, wherein the alert is one or more of an audio alert, a visual alert, a tactile alert, and a textual alert.

8. The system of claim 1, further comprising a plurality of pressure sensors each sensor configured to contact one or more surfaces of the foot, wherein the edema detection application is further configured to:
detect a plurality of transient increases and subsequent decreases in the detected pressure from the plurality of pressure sensors;
determine a plurality of pressure rebound curves based on changes in the pressure following the plurality of transient increases and the subsequent decreases in the detected pressure;
determine a combined pressure rebound curve based on an average of the plurality of pressure rebound curves;
compare the combined pressure rebound curve to the normal pressure rebound curve; and
in response to detecting that the combined pressure rebound curve indicates a slower increase rate in the detected pressure than a pressure increase rate indicated by the normal pressure rebound curve, infer presence of the foot edema.

9. The system of claim 8, wherein the edema detection application is further configured to:
weight one or more of the plurality of pressure rebound curves to determine the combined pressure rebound curve.

10. The system of claim 9, wherein the edema detection application is further configured to:
weight the one or more of the plurality of pressure rebound curves based on locations of one or more corresponding pressure sensors.

11. The system of claim 1, further comprising a plurality of pressure sensors each sensor configured to contact one or more surfaces of the foot, wherein the edema detection application is further configured to:
detect a plurality of transient increases and subsequent decreases in the detected pressure from the plurality of pressure sensors;
determine a plurality of pressure rebound curves based on changes in the detected pressure following the plurality of transient pressure increases and the subsequent decreases; and
select a pressure rebound curve of the plurality of pressure rebound curves that indicates a fastest rebound in the detected pressure as the first pressure rebound curve.

12. A method to detect foot edema through pressure sensing, the method comprising:
detecting a first transient increase and a subsequent decrease in a pressure detected through a pressure sensor in contact with a foot, wherein the pressure is generated on a surface of the foot by fluid accumulation within cells of the foot;
monitoring a change in the pressure following the transient increase and the subsequent decrease in the detected pressure to determine a first pressure rebound curve;
in response to detecting expiration of a predefined time period following the detection of the first transient increase and the subsequent decrease in the detected pressure, inferring a rest state;
in response to inferring the rest state, comparing the first pressure rebound curve to a normal pressure rebound curve, wherein the normal pressure rebound curve corresponds to a change in detected pressure of a healthy person's foot; and
in response to detecting that the first pressure rebound curve indicates a slower increase rate in the detected pressure than a pressure increase rate indicated by the normal pressure rebound curve, infer presence of the foot edema.

13. The method of claim 12, further comprising:
in response to inferring the presence of the foot edema, transmitting one or more of an audio alert, a visual alert, a tactile alert, and a textual alert to a computing device.

14. The method of claim 12, further comprising:
determining that one or more pressure rebound curves indicate a faster increase rate in the detected pressure than the pressure increase rate indicated by the normal pressure rebound curve in the rest state; and
calibrating the normal pressure rebound curve based on the one or more pressure rebound curves.

15. The method of claim 12, further comprising:
adjusting the fast pressure rebound curve based on one or more parameters associated with a person on whose foot the first transient increase and the subsequent decrease is detected.

16. The method of claim 15, wherein the one or more parameters include one or more of: a weight of the person, a size of the foot of the person, an average blood pressure level of the person, and a health indicator of the person.

17. The method of claim 12, further comprising:
detecting a plurality of transient increases and subsequent decreases in the detected pressure through a plurality of pressure sensors in contact with the foot;
determining a plurality of pressure rebound curves based on changes in the pressure following the plurality of transient increases and the subsequent decreases in the detected pressure;
determining a combined pressure rebound curve based on a weighted average of the plurality of pressure rebound curves, wherein the one or more of the plurality of pressure rebound curves are weighted based on locations of corresponding pressure sensors;
comparing the combined pressure rebound curve to the normal pressure rebound curve; and
in response to detecting that the combined pressure rebound curve indicates a slower increase rate in the detected pressure than the pressure increase rate indicated by the normal pressure rebound curve, inferring presence of the foot edema.

18. An edema detection device configured to detect foot edema through pressure sensing, the edema detection device comprising:
a communication module communicatively coupled through a wired or wireless communication medium to one or more pressure sensors each sensor configured to contact a foot and detect a pressure generated on a surface of the foot by fluid accumulation within cells of the foot;
a memory configured to store instructions; and
a processor coupled to the memory and the communication module, the processor configured to:
detect one or more transient increases and subsequent decreases in the detected pressure through the one or more pressure sensors;
determine a pressure rebound curve with each of the one or more pressure sensors based on changes in the pressure following the one or more transient increases and subsequent decreases in the detected pressure;
determine a combined pressure rebound curve based on a weighted average of the pressure rebound curves, wherein the pressure rebound curves are weighted based on locations of corresponding pressure sensors;
compare the combined pressure rebound curve to a normal pressure rebound curve, wherein the normal pressure rebound curve corresponds to a change in pressure of a healthy person's foot;
in response to detecting that the combined pressure rebound curve indicates a slower increase rate in the detected pressure than a pressure increase rate indicated by the normal pressure rebound curve, infer presence of the foot edema; and
transmit one or more of an audio alert, a visual alert a tactile alert, and a textual alert to a computing device.

19. The computing device of claim 18, wherein the edema detection device is portable device attachable to a shoe.

20. The computing device of claim 18, wherein the edema detection device is integrated with a shoe.
